(19) United States
(12) Patent Application Publication
(10) Pub. No.: US 2021/0077732 A1
(43) Pub. Date: Mar. 18, 2021

(54) READY-TO-USE CRYORESISTANT INJECTION DEVICE

(71) Applicant: ÉTABLISSEMENT FRANÇAIS DU SANG, Saint-Denis (FR)

(72) Inventor: Harald EGELHOFER, Corenc (FR)

(73) Assignee: ÉTABLISSEMENT FRANÇAIS DU SANG, Saint-Denis (FR)

(21) Appl. No.: 16/772,229

(22) PCT Filed: Dec. 14, 2018
(86) PCT No.: PCT/FR2018/053298

§ 371 Date: Jun. 12, 2020

(30) Foreign Application Priority Data
Dec. 14, 2017 (FR) 1762146

(51) Int. Cl. A61M 5/31; A61M 5/315; A61M 5/32

(52) U.S. Cl. CPC A61M 5/3134; A61M 5/315; A61M 2005/312; A61M 2005/311; A61M 2005/3131; A61M 5/3202

(57) ABSTRACT

A sealed and cryoresistant device for injecting a solution, the device being designed to be associated with a needle for subsequent injection of the solution and including an injection body, a plug and a plunger head. The injection body includes a first, proximal end closed in a sealed manner by the plunger head and a second, distal end closed in a sealed manner by the plug. The plug is held on the injection body by a fastening and includes a breakable portion allowing a needle to be mounted, the plug being a divisible plug, the separation of a divisible portion releasing an element for fastening an injector including a needle. The materials forming the device are cryoresistant.

### FIELD OF THE INVENTION

The present invention concerns a device for injecting a cold-preserved liquid form, such as in particular frozen biological products or drugs of animal or human origin. More particularly, the present invention concerns the field of syringe-type injection devices, in particular those that are ready-to-use.

### PRIOR ART

Certain pharmaceutical products need to be preserved frozen, or even cryogenically stored, in order to ensure good preservation of their pharmacological and pharmacodynamic properties. In order to be frozen or cryogenically stored, they are often placed in ampoules, pouches, vials or capsules containing a dose to be administered. This is the case, in particular, for products comprising cells. For these fragile products, it is not intended to be necessary to unpackage the product after thawing or removal from cryogenic storage, in order to then be injected. This type of manipulation generates risks, such as contamination, cell loss, etc.

There is therefore a need for a ready-to-use cryoresistant packaging minimising the risks of contamination and reducing the number of manipulations.

WO 2016/019108 describes a cryoresistant vial comprising a tube and two plugs situated at each end, which frees a space for a plunger and a needle. The vial can therefore be converted into a syringe. However, the unpackaging of the device requires many manipulations, in particular the unscrewing of the plugs and the mounting of a plunger, a hub and a needle. Finally, this solution cannot ensure that a preceding manipulation has not been carried out on the vial, which does not guarantee maximum security with respect to a possible contamination of the solution.

Similarly, WO 2007/044980 describes a device for injecting a dose of a drug and/or cells stored at a temperature below −40° C. The device comprises modular elements in order to deliver a thawed solution to be administered by a syringe after several manipulations. In particular, it is necessary to transfer the thawed liquid into a reservoir of the syringe. The device described in WO 2007/044980 therefore imposes a manipulation of the liquid to be administered from one chamber to another for its administration. The disadvantage of this solution is that it involves a device requiring manipulations exposing the liquid to risks of contamination after thawing before obtaining a syringe that is ready to use. Finally, another disadvantage results from the many manipulations requiring caution and vigilance to be maintained by an operator during each of the syringe preparation steps. Whatever the envisaged manipulations may be, they require an environment suitable for reducing their inherent risks.

Document EP 2 253 349 describes a cryoresistant syringe for injecting a liquid drug, comprising a cylindrical body, a plug having a sealing member, and a plunger. The described syringe imposes additional manipulations before injection of the drug, since the sealing member needs to be withdrawn from the plug before fitting the needle on the cylindrical syringe barrel.

Document EP 2 554 205 describes a cryoresistant medical device for injection of a medical agent solution. This device comprises an outer cylinder, a plug and a plunger head. The plug comprises a portion which can be impacted by a needle in order to allow the injection of the solution contained in said device. This solution proves fragile since the impactable portion can be pierced inadvertently and accidentally before use of the device.

Document WO 2009/086829 describes a breakable storage container allowing the release of a solution after fitting of the needle. The container comprises a tubular body and two plugs closing each of the ends of the tubular body. The body of the container is breakable in a plurality of locations in order to release the solution contained in said body, making it particularly fragile. Moreover, the container does not include a plunger for injection of the solution.

Document US 2006/019233 describes an apparatus for cryogenic storage of biological materials such as a sealed syringe enabling the injection of a solution. The syringe comprises a body, a first plug and a plunger head on which a plunger is fitted. The described apparatus imposes additional manipulations before injection of the biological material, since the plug needs to be withdrawn before fitting the needle on the cylindrical syringe barrel.

### SUMMARY OF THE INVENTION

The invention allows the above-described disadvantages of the prior art to be solved. In particular, the injection device according to the invention can respond to these two requirements of a device that is ready-to-use and which is cryoresistant. One of the many advantages of the invention is therefore to enable administration of a thawed liquid form while minimising the number of manipulations and therefore the risks (leaks, contamination, cell loss). The invention also aims to propose a non-reusable disposable device, which keeps the solution safe by means of the closure of the two ends of the injection body.

According to a first aspect, the invention concerns a cryoresistant device for injecting a solution, this device comprising an injection body, a plug and a plunger head; the injection body comprises a first proximal end, closed in a sealed manner by the plunger head, and a second, distal end closed in a sealed manner by the plug; the plug is held to the injection body by a fastening and comprises a breakable portion releasing a hub enabling the mounting of a needle. The plug comprising a breakable portion is a divisible plug, the separation of a divisible portion releasing a means of fastening an injector comprising a needle.

Due to the presence of a closed plug, for which a rupture (or break) must be engaged, the risk of loss of contents and the risk of a leak is avoided. Moreover, the solution of the invention enables an increase in security due to the presence of a closed plug for which a divisible element ensures the sealing and insulation. The solution of the invention hence allows a maximum level of security.

The solution also offers an economic gain due to the possibility of using a conventional injector and time savings with respect to other systems.

The presence of a plunger head enabling the sealed closure of the first proximal end of the injection body makes it possible to avoid the risk of leaks at this end and to preserve the solution intact inside the injection body.

This double closure hence allows a maximum level of security.

According to an embodiment, the device according to the invention further comprises a seal closing the first proximal end of the injection body in a sealed manner. The seal is added to the plunger head in order to ensure a fully hermetic closure of said first proximal end of the injection body. It also allows the plunger head to be protected against any unforeseen manipulation. It can be made, for example, from aluminium.

According to an embodiment, all the components of the device according to the invention are made from a cryoresistant material. By way of example, the injection body can be made from polypropylene. Also by way of example, the plunger (head, rod and/or thumb press) and/or gaskets can be made of silicone, of PTFE (polytetrafluoroethylene) or of a suitable TPE (thermoplastic elastomer) with a hardness appropriate to each use. An advantage of PTFE it is to be particularly suitable for very low temperatures, in particular for its use with mechanical parts.

According to an embodiment, the means for fastening an injector to the plug is a hub suitable for mounting a needle.

According to an embodiment, the hub of the plug comprises a fitting means such as a thread or a guide arranged on a circumferential portion. This thread is suitable for cooperating with a hub of an injector comprising a needle, said hub comprising a complementary fitting means suitable for an inner or outer circumferential portion. The fitting means may be a thread, a guide such as a circular rail, or any other form enabling fitting to a complimentary form of the hub of the plug. According to an embodiment, the hub of the plug is of Luer Lock type.

According to an embodiment, the fitting means, such as a thread, can be arranged on the inner surface of the hub of an injector.

The divisible portion is a divisible end piece.

According to this embodiment, the divisible plug comprises a base and a divisible end piece which can, in an embodiment, form a rod extending said base; the base is integral with the injection body; in an embodiment, the divisible plug comprises further comprises a filter for filtering the liquid form to be injected.

According to an embodiment, the base or the periphery of the base of the divisible plug is integral with, preferably welded to, one end of the injection body. These various embodiments advantageously demonstrate that the invention considerably limits the risk of external contamination. According to an embodiment, the base or the periphery of the base of the divisible plug can be made integral with one end of the injection body by definitive snap-fitting, or any other means known to a person skilled in the art. Definitive snap-fitting means that once snap-fitted on an end of the injection body, the base cannot be removed from said end.

According to an embodiment of the invention, the plug comprises a cryoresistant gasket.

According to an embodiment of the invention, the breakable plug is cryoresistant down to −196° C.

According to a second aspect, the invention concerns an injection assembly comprising: (i) a cryoresistant device according to the invention, for injecting a solution, comprising a breakable plug, (ii) an injector (iii) a plunger rod and (iv) a plunger thumb press; said injector comprising a needle, a needle support, a hub integral with the support and fitting to the plug of the device according to the invention after severing of the divisible portion. The plunger rod and the plunger thumb press are designed to cooperate with the plunger head of the device.

An advantage of the invention is to provide a cryoresistant device which is independent of the injector. It is therefore not necessary to store the cryoresistant device with the injector, which leads to a gain in storage space of the solutions to be cryogenically stored.

According to an embodiment, the injector comprises a protective cap, and the needle comprises a single or multiple bevel at the distal end thereof, and/or a single or multiple bevel at the proximal end thereof, intended to be placed on the hub of the divisible plug after separation of the divisible portion.

According to a third aspect, the invention concerns a method for preparing a disposable ready-to-use cryoresistant device, being a method for preparing a liquid form to be cryogenically stored.

The method comprises the following steps:
introducing a liquid form into the device of the invention, made up of the injection body, the plunger and a seal which has preferably been assembled under sterile conditions;
closing the injection body by the fastening of the divisible plug;
freezing or cryogenic storage of the device.

According to a fourth aspect, the invention concerns a method for preparing a liquid form to be injected, comprising the following steps:
thawing of a liquid form contained in an injection device of the invention;
removal of the protective seal of the injection body and fitting of a plunger rod and a plunger thumb press on the plunger head of the device;
mounting an injector suitable for the plug of the device, after severing the divisible plug.

### Definitions

In the present invention, the terms below are defined in the following manner:
“cryoresistant” refers to a part or a solution that is resistant to cold and in particular to very low temperatures, for example less than 150° C.
“lug” designates a projection at the surface of a manufactured object, which allows a holding stop or a retention stop of a mechanical part to be formed.
“seal” designates a protective element, usually forming a consumable element. It may be, for example, a cover or a protective pellet.
“bevel” designates a profile forming the end of a sharp or piercing object. A bevel can generally be produced by an inclination of the end of the object, such as a needle. Reference is made in general to the sharp or piercing part of a sharpened tool.

### BRIEF DESCRIPTION OF THE FIGURES

FIG. 1 illustrates an example injector capable of cooperating with a plug according to the first embodiment or the second embodiment.

FIG. 2 illustrates a plunger comprising a thumb press, a rod and a head.

FIG. 3 illustrates an example of a plug comprising a divisible end piece according to a second embodiment.

FIG. 4 illustrates an example of a syringe associated with a plug according to the invention.

FIG. 5 illustrates an example of a filter cooperating with the plug of FIG. 3

FIG. 6 illustrates the syringe barrel of FIG. 5, comprising a sealing element for the preservation of the syringe.

### REFERENCE SIGNS

1: syringe barrel
10: barrel flange surface
101: seal of the syringe barrel cooperating with the barrel flange surface
21: plug intended to be associated with a syringe barrel
211: divisible portion or element
212: base of the plug 21, capable of cooperating with the distal end of the syringe barrel
213: filter
210: hub
3: plunger
30: plunger thumb press
31: plunger head
32: plunger rod
4: injector
41: needle
410: second bevel of the needle 41
411: first bevel of the needle 41
42: needle support
43: needle hub
45: cap

### DETAILED DESCRIPTION

According to one aspect, the invention concerns a plug 21 intended to be associated with a syringe barrel 1. In the description which follows, the terms “injection device” and “syringe” are used interchangeably. “Injection assembly” shall mean the injection device for the syringe with which an injector 4 is associated. Similarly, in the description which follows the terms “injection body” and “syringe barrel” are used interchangeably.

The plug 21 is intended to be integral with the syringe barrel 1 with the aim of holding and preserving a solution to be administered, in a sealed manner. In an embodiment, the plug 21 can have a diameter substantially equal to the diameter of the syringe barrel 1.

When the syringe is ready to be used after a period of preservation, the plug 21 comprises a breakable portion for releasing a hub 210. The hub 210 can then be associated with an injector 4 in order to inject the solution.

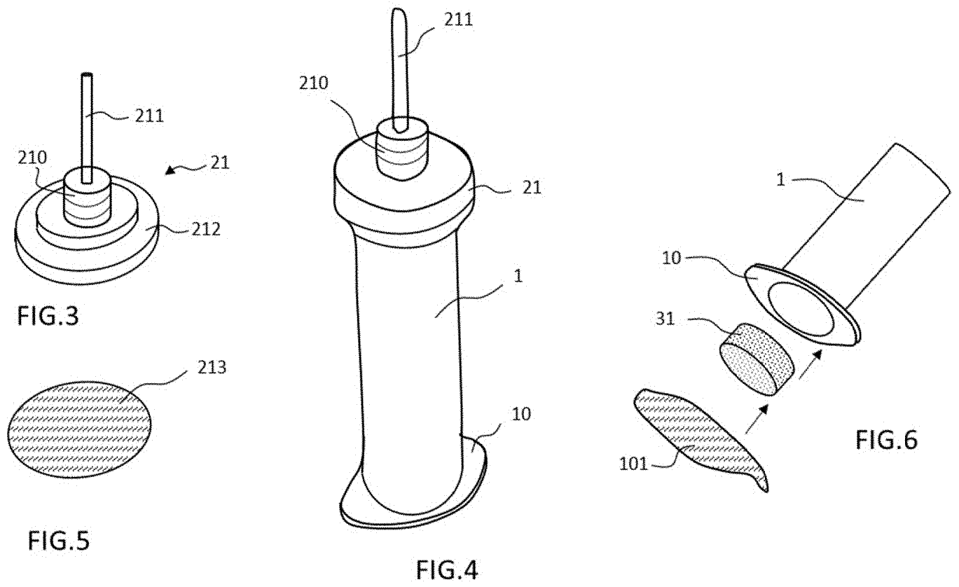

An embodiment describes a plug 21 comprising a divisible element 211, this embodiment is described in particular by means of FIGS. 3, 4, 5 and 6.

The plug 21 does not comprise an impactable element able to be impacted or pierced by a needle.

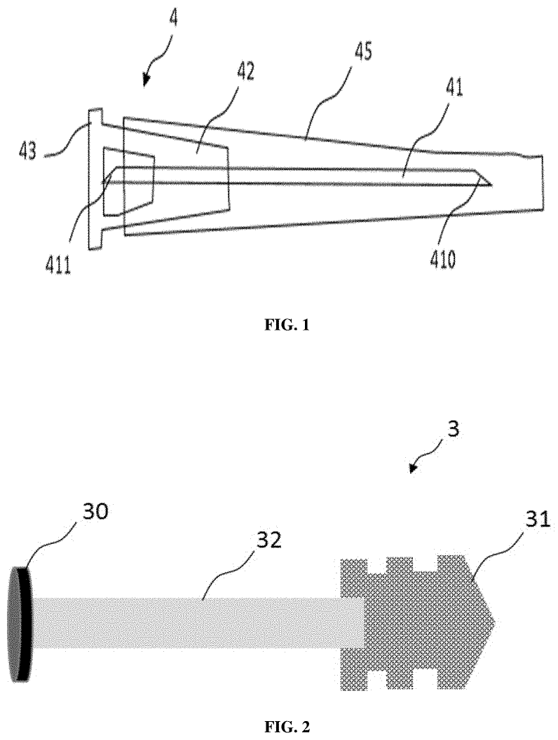

The injector 4 of FIG. 1 is compatible with the breakable plug 21 if the various possible interactions between the hub 43 of the injector 4 and the hub 210 of the plug 21, for example of “Luer Lock” type, are considered. Other hubs can be used according to other embodiments, from the moment when the injector 4 comprises a fastening allowing it to secured to the syringe (also called the injection device).

According to an exemplary embodiment, the body 1 is made of polymer, such as polypropylene or cyclic olefin copolymer (COC), which makes it possible to ensure a cryoresistance down to at least a temperature of −130° C., −140° C., −150° C., −160° C., −170° C., −180° C., −190° C. or −196° C. The body 1 is preferably cryoresistant down to at least a temperature of −196° C. An advantage of COC is that it is a transparent cryoresistant material.

According to an embodiment, the plug 21 is made of polymer, such as polypropylene or cyclic olefin copolymer (COC). The breakable plug 21 is cryoresistant down to at least a temperature of −130° C., −140° C., −150° C., −160° C., −170° C., −180° C., −190° C. or −196° C. The plug 21 is preferably cryoresistant down to at least a temperature of −196° C. An advantage of COC is that it is a transparent cryoresistant material.

According to an exemplary embodiment, the elements of the device in contact with the solution to be injected comprise at least one biocompatible material, free of heavy metals, material of animal origin or salting-out chemicals.

FIG. 1 illustrates an injector 4 which is associated with the injection body 1. According to an embodiment, the injector 4 comprises a needle 41, a needle support 42 designed to hold the needle 41, and a hub 43. According to an exemplary embodiment, the hub 43 is integral with the needle support 42. According to an example, the support 42 and the hub 43 are designed in a same material and form a one-piece part.

According to an embodiment, the hub 43 comprises an external thread in order to cooperate with the internal thread of the Luer-Lock end piece. According to other embodiments, the hub 43 of the injector 4 comprises a fastening means able to be integral with an end piece of the plug 21.

The first end of the needle 41 is arranged substantially in the centre of the hub 43 and is driven with translational movements of the injector 4 when the latter is fitted on the injection body 1.

The needle 41 comprises, at its second end, a second bevel 410 in order to offer optimum penetration.

The needle 41 comprises, at its second end, a second bevel 410 in order to offer a better penetration of the needle 41 into the skin.

According to an embodiment, the injector 4 comprises a cap 45 in order to protect the needle 41. The needle 41 is not presented directly in this case, it is necessary to remove the cap 45. Moreover, the cap 45 makes it possible to fasten the injector 4 to the plug 21 in a simple manner, for example by holding the cap 45 between the fingers. The injector 4 can be screwed, snap-fitted or clamped on the plug 21.

Moreover, the cap 45 makes it possible to avoid contamination of the needle 41.

According to an embodiment, the plug 21 comprises a Luer Lock end piece. According to an example, the end piece comprises an internal thread cooperating with an external thread of a hub 43 of an injector 4. According to an example, the end piece is a Luer-Lock end piece of standard ISO 594/1-1986, NF EN 20594-1:1993-12.

According to an embodiment, the injector 4 comprises a bevelled double-inlet needle 41. According to an embodiment, the injector 4 comprises a bevelled single-inlet needle 41, this single inlet being used for the injection.

FIG. 3 illustrates a plug 21 according to a second embodiment. The plug 21 comprises a divisible portion 211. According to an exemplary embodiment, the divisible portion 211 forms a hollow or solid rod extending over a portion of several centimetres. Hence the severing of the breakable portion can be facilitated and can be performed by bending undertaken with the fingers. In the case where the rod is hollow, it is hermetically closed at its end furthest away from the injection body 1.

According to an example, the rod comprises at least one fragility region on a circumferential portion so as to cause a braking in a predetermined region. According to an embodiment the fragility region is a notch with a circumferential portion or groove. According to an embodiment, the notch or groove is produced so that the fracturing, breaking or rupture is generated at the base of the rod at its junction with the hub 210. The notch or groove creates a breaking point enabling easy breaking of the rod. The notch or groove enables uniform breaking over the entire circumference of the rod, and avoids the production of debris resulting from the break.

According to an embodiment, the notch is a groove applied by a pre-filing of the rod. According to an embodiment, the pre-filing is carried out over the entire perimeter of the rod, or over a portion of the perimeter of the rod, so that the notch or groove extends over the entire perimeter of the rod or over a portion of the perimeter of the rod.

According to an embodiment, the notch is produced by a “colour break” process which consists of depositing a material having a coefficient of expansion different from that of the material of the rod at the place where the rod should break.

According to an embodiment, the notch is produced by an “Anrep” process which consists of heating the material of the rod in a very localised manner in order to generate internal stresses in the material of the rod and, consequently, to weaken it.

According to an embodiment, the notch or groove is produced by any method known to a person skilled in the art

According to an embodiment, the rod is broken by the application of a rupture force consisting of a torque from forces in opposite directions and perpendicular to the direction of the rod. The rod is held stationary during the application of said rupture force.

According to an embodiment, the rod is self-breakable, in other words it is not necessary to use any tool, such as a file for example, in order to break the rod.

According to an embodiment the rod is a tube, a cylinder of circular or ovoid cross section, or a parallelepiped.

According to an embodiment, the rod has a length ranging from 0.5 cm to 5 cm.

According to an embodiment, the rod has a diameter ranging from 0.5 mm to 5 mm.

According to an embodiment, the rod has a side section ranging from 0.5 mm to 5 mm.

According to an embodiment, the rod is made of polymer, such as polypropylene or cyclic olefin copolymer (COC) which makes it possible to ensure cryoresistance down to at least a temperature of −130° C., −140° C., −150° C., −160° C., −170° C., −180° C., −190° C., or −196° C. The rod is preferably cryoresistant down to at least a temperature of −196° C.

According to an exemplary embodiment, the rod is solid over an upper part and hollowed out over a lower part of the plug 21 so that the injector 4 can sample a portion of the solution even if the rod is fractured a little higher up than the base of its junction with the hub 210.

According to an embodiment, the plug 21 comprises a base 212 capable of cooperating with the proximal end of the syringe barrel 1.

According to an exemplary embodiment, the plug 21 is definitively fastened to the syringe barrel 1.

According to an exemplary embodiment, the plug 21 is welded to the syringe barrel 1.

According to an exemplary embodiment, the plug 21 is definitively snap-fitted to the syringe barrel 1.

According to an exemplary embodiment, the fastening of the plug 21 on the injection body 1 comprises at least one tamper-protection lug arranged on a peripheral portion of the injection body 1.

According to an exemplary embodiment, the plug 21 advantageously comprises a circumferential lug forming an element for holding the plug 21 on the injection body 1 when it is snap-fitted to the contact of a snap-fitting lug projecting from the surface of the injection body 1. The two lugs have complementary geometries allowing the formation of an assembly forming an action and an opposing reaction in order to secure the plug 21 to the injection body 1.

In an embodiment, the snap-fitting plug 21 is fastened to the injection body 1. The seal is then ensured by the presence of the circumferential gasket and the holding lug which holds the joint in compression.

Advantageously, the plug 21 comprises an edge extending over a slight portion of the outer surface of the syringe barrel 1.

According to an embodiment, the edge which extend longitudinally along the injection body 1. This edge makes it possible to reinforce the body-plug connection.

According to an exemplary embodiment, the holding lug is positioned at the end of this edge. According to an example, the lug only covers a part of the inner circumference of the plug 21. According to an exemplary embodiment, the lug present on the injection body 1 also partially covers the circumference of said body 1. According to another exemplary embodiment, the two lugs are snap-fitted to each other by deforming the plug 21 during its fitting on the injection body 1.

According to an embodiment, the plug 21 comprises a hub 210 having a thread arranged on its outer surface. The hub 210 is preferably of the Luer Lock connector type. The thread 210 cooperates with the thread of the injector 4. According to an exemplary embodiment, the hub 43 of the injector 4 comprises an internal thread in order to cooperate with the thread of the hub 210.

According to one aspect, the invention concerns an injector 4 such as that illustrated in FIG. 1 comprising a hub 43 having a means for fitting to a connector, for example of the Luer Lock type. According to an example, the hub 43 comprises an external thread and an internal thread. According to an embodiment, the injector 4 also comprises a seal (not illustrated) closing the hub 43 which needs to be removed before the fastening of the injector 4 on the plug 21.

FIG. 4 illustrates a syringe barrel 1 with which a plug 21 is associated. According to an exemplary embodiment illustrated in FIG. 5, a filter 213 it is introduced between the plug 21 and the syringe barrel 1 so as to filter the microaggregates and/or microparticles of the liquid form which will be injected. Such a filter provides increased security with respect to the liquid form to be administered, which may for example contain a suspension comprising microaggregates and/or microparticles. According to an embodiment, the filter 213 is conical, which enables, in particular, an optimum shape to be obtained fitting to the plug 21. According to another example, it is flat.

According to an embodiment, the plug 21 also comprises an inner gasket forming a contact between the plug 21 and the syringe barrel 1. According to an embodiment, the plug 21 is welded on the syringe barrel 1. Thus, the sealing of the syringe barrel 1 can be ensured by the fastening of a plug 21 on the syringe barrel 1 with, for example, a gasket and a weld.

According to an embodiment illustrated in FIG. 4, the syringe barrel 1 comprises an end forming a barrel flange surface 10. The barrel flange surface 10 forms a circumferential rim for ergonomic holding during the injection. In addition, the barrel flange surface 10 allows a support zone to form when a seal 101 is glued as illustrated in FIG. 6. The seal 101 cooperates with the barrel flange surface 10.

According to an embodiment, the plunger 31, formed by a rod 32 and a thumb press 30, is used after thawing of the injection device (FIG. 2). According to an embodiment, a plunger head 31 can be inserted in the syringe barrel 1 before freezing or cryogenic storage of the device, and can comprise means for fitting to a rod 32 and a thumb press 30 that are not illustrated in FIG. 6. The plunger head 31 and the seal 101 can therefore be incorporated in the syringe barrel 1 beforehand in order to ensure a good sealing of the syringe barrel 1. After thawing the device, the seal 101 is removed, thus releasing the distal end of the injection body 1, and the rod 32 and the thumb press 30 can be fitted on the plunger head 31 in order to form a plunger 3. This solution allows the solution to be preserved in a sealed manner while providing a ready-to-use syringe when it is thawed.

According to an exemplary embodiment, the seal 101 can comprise a barcode or a 2D code including an indication of the product contained in the syringe barrel 1. According to another example, the seal 101 includes information on the type of injector 4, rod 32 and/or thumb press 30 to be used on the diameter of the hub 43 which should be used.

The seal 101 makes it possible, in particular, to protect the plunger head 31 and the solution to be injected, and to hold them under conditions which avoid any external contamination or loss of solution (leaks).

According to an embodiment, the seal 101 is made of aluminium. According to an exemplary embodiment, they are glued or welded to the outside of the syringe barrel 1.

According to an embodiment, the seal 101 has a diameter substantially greater than that of the injection body 1.

According to an exemplary embodiment, a tab can be used on a seal 101 obstructing the proximal end of the injection body 1.

According to an exemplary embodiment, the plunger comprises a thumb press 30 or a thumb press element for driving the plunger 3 in translation inside the injection body 1. The plunger 3 further comprises a rod 32 for driving a head 31 forming a sealing element in order to avoid any leak of the solution contained in the injection body 1. The head 31 preferably has a circumference suitable for moving along the inner wall of the injection body 1 while forming a sealed wall.

According to an embodiment, the plunger head 31 can be detached so as to form a plunger 3 without rod and without thumb press. One advantage is to allow the solution to be preserved with a plunger head 31 intended to cooperate with a rod 32. A junction 301 enables the rod 32 to penetrate into the plunger head 31.

The advantage of not having the rod 32 or the thumb press 30 of the plunger 3 in the device to be cryogenically stored or frozen, is to limit the quantity of material that needs to be cryoresistant. Hence, the thumb press 30 and the rod 32 can be consumables installed at the time of thawing the device and the solution contained in the injection body 1.

According to an example, the plug 21 is sealed and can retain the liquid form in the syringe barrel 1.

According to another example, components are produced according to the invention from medical grade raw materials in compliance with the USP (Class IV), the European Pharmacopoeia and standard ISO 10993.

According to an embodiment, a cryoresistant material (0° C. down to at least −196° C., whatever the refrigeration means) can be used that is autoclavable according to standard protocols described in the European Pharmacopoeia.

According to an embodiment, a geometry of the Luer-lock connection can be determined according to standard NF EN 20594-1:1993-12.

According to an embodiment, the size and/or volume of the variable constituent elements can be chosen, according to need, from 2 to 60 ml.

According to an embodiment, the injection body 1 has a cylindrical shape.

According to an embodiment, the injection body 1 has a volume between 2 and 60 ml.

According to an embodiment, the injection body 2 has a length between 4 cm and 15 cm.

According to an embodiment, the injection body 2 has a diameter between 0.5 cm and 5 cm.

According to various embodiments, the components of the injection device and/or the injection assembly could be packaged together or separately in a cryoresistant packaging or double packaging capable of insulating the injectable contents from the immediate environment.

The invention also concerns a method for preparing a liquid form to be frozen/cryogenically stored.

According to an embodiment of the invention, the preparation method of a liquid form to be cryogenically stored comprises the following steps:
introducing a liquid form into the device of an embodiment of the invention, made up of the injection body 1, the plunger head 31 and the seal 101 which have been assembled beforehand, preferably under sterile conditions;
closing the injection body 1 by the fastening of a breakable plug 21;
freezing or cryogenic storage of the device.

In an embodiment, the liquid form is a solution. The terms “liquid form” and “solution” can be used interchangeably in the present description.

According to another example, the plug 21 comprises a breakable element of the divisible type.

According to an embodiment, this step of introducing the liquid form into the device is carried out using a manual or automated filling device for tube filling.

According to an embodiment, the method comprises a preliminary step of assembling the injection body 1, the plunger head 31 and the seal 101, during which the plunger head 31 is introduced into the injection body 1 via its proximal end and the seal 101 is then glued in order to seal said proximal end of the injection body 1.

According to an embodiment, the step of closing the injection body 1 by the fastening of a breakable plug 21 consists of closing said injection body 1 by welding, or definitive snap-fitting of the plug 21 on the distal end of the injection body 1.

According to an embodiment, the step of freezing or cryogenic storage of the injection device is carried out using the most appropriate means for the liquid form. If the liquid form comprises a biological product, this corresponds to the most appropriate means for the biological product.

According to an embodiment, the step of freezing or cryogenic storage of the injection device comprises a step of programmed freezing with a temperature step of −1.5 to −2.5° C./min.

According to an embodiment, the step of freezing the injection device obtaining a liquid form comprises the cooling of the said device to a temperature of at least −120° C.

According to an embodiment, the step of cryogenic storage of the injection device containing a liquid form comprises the cooling of said device to a temperature of at least −196° C. This step is carried out using liquid nitrogen or nitrogen vapour in which the device is immersed.

The invention also concerns a method for preparing a liquid form to be administered by injection.

According to an embodiment, the preparation method of a liquid form to be injected comprises the following steps:
thawing of a liquid form contained in an injection device of the invention;
removing the protective seal 101 of the injection body 1;
severing the breakable plug 21;
fastening an injector 4 comprising a hub 43 fitting to the plug 21 of the device of the invention.

The injector 4 can be fastened using a thread of a hub 210 of an end piece of the plug 21 for example, of a Luer-lock type element.

The severing of the breakable plug 21 corresponds to a fracturing of a divisible element 211. Once the divisible element 211 is removed, a hub 210 comprising a thread is released in order to fasten an injector 4.

The fastening of the injector 4 is simple, quick and requires few manipulations. The device according to the invention enabling this fastening has the advantage of allowing a direct injection of the solution to be injected. It is therefore not necessary to unpackage the liquid form, to wash it and to repackage it before injection

According to an embodiment, the method further comprises, after the step of removing the seal 101, a step of fitting a rod 32 and a thumb press 30 on the plunger head 31 in order to produce a complete plunger 3. During this step, the volume of liquid form to be injected can be adjusted using the rod of the plunger 32 bearing graduations.

According to an embodiment, the method further comprises a last step, being a step of injecting the liquid form. During this step, the liquid form can be injected, for example, into the body of a subject, into a pouch, vial, container, culture medium or any medium or any type of container known to a person skilled in the art.

1-10. (canceled)

11. A sealed and cryoresistant device for injecting a solution, said device being designed to be associated with a needle for subsequent injection of said solution and comprising an injection body, a plug and a plunger head, wherein:
the injection body comprises a first proximal end closed in a sealed manner by the plunger head and a second, distal end closed in a sealed manner by the plug;
the plug is held on the injection body by a fastening and comprises a breakable portion releasing a hub allowing a needle to be mounted, the plug being a divisible plug, the separation of a divisible portion releasing a means for fastening an injector comprising a needle; and
the materials forming the device are cryoresistant.

12. The device according to claim 11, wherein the means for fastening an injector to the plug is a hub suitable for mounting a needle.

13. The device according to claim 11, wherein the divisible plug comprises a base and a divisible end piece forming a rod extending said base, said base being integral with the injection body, the divisible plug further comprising a filter for filtering the solution to be injected.

14. The device according to claim 13, wherein the edge of the base of the divisible plug is integral with the end of the injection body.

15. The device according to claim 11, wherein the device comprises a seal closing, in a sealable manner, the first proximal end of the injection body.

16. An injection assembly comprising a cryoresistant device according to claim 11, an injector, a plunger rod and a plunger thumb press, said injector comprising a needle, a needle support, a hub integral with the support and fitting to the plug of the device, the rod and the plunger thumb press being designed to cooperate with the plunger head of the device.

17. The injection assembly according to claim 16, wherein the injector comprises a protective cap, and the needle comprises a multiple bevel at the distal end and a multiple bevel at the proximal end intended to be placed on the hub of the divisible plug after separation of the divisible portion.

18. A method for preparing a liquid form to be cryogenically stored, comprising the following steps:
introducing a liquid form into the device according to claim 11;
closing the injection body by the fastening of the breakable plug; and
cryogenic storing of the device.

19. A method for preparing a liquid form to be injected, comprising the following steps:
thawing a liquid form contained in a device according to claim 11;
removing the protective seal of the injection body;
severing the breakable plug; and
fastening an injector comprising a hub fitting to the plug of the device.

20. The method according to claim 19, wherein the method comprises a step of fitting a plunger rod and thumb press on the plunger head of the device.
